(19) United States
(12) Patent Application Publication
(10) Pub. No.: US 2020/0087713 A1
(43) Pub. Date: Mar. 19, 2020

(54) NUCLEIC ACID QUANTIFICATION BY TEMPORAL BARCODING

(71) Applicant: The Board of Trustees of the Leland Stanford Junior University, Stanford, CA (US)

(72) Inventor: Julia Salzman, Palo Alto, CA (US)

(21) Appl. No.: 16/567,835

(22) Filed: Sep. 11, 2019

Related U.S. Application Data
(60) Provisional application No. 62/732,344, filed on Sep. 17, 2018.

(51) Int. Cl. C12Q 1/6818; C12Q 1/6804; C12Q 1/6827; C12N 15/10; C40B 70/00

(52) U.S. Cl. CPC C12Q 1/6818; C12Q 1/6804; C12Q 2537/143; C12N 15/1065; C40B 70/00; C12Q 1/6827

(57) ABSTRACT

This disclosure provides, among other things, a method of analyzing a sample by hybridizing oligonucleotides in a predefined temporal order. In some embodiments, the method may comprise: sequentially hybridizing multiple sets of barcoded detector oligonucleotides with a sample that comprises a population of target molecules, wherein the detector oligonucleotides are complementary to a sequence in the target molecules and the different sets of oligonucleotides hybridize to the sequence in a predefined temporal order, and wherein the barcodes of the detector oligonucleotides identify the order of hybridization; and quantifying the amount of each barcode sequence in the barcoded detector oligonucleotides that hybridize to the sequence in the population of molecules.

### CROSS-REFERENCING

This application claims the benefit of U.S. provisional Application No. 62/732,344, filed on Sep. 17, 2018, which application is incorporated by reference herein in its entirety.

### BACKGROUND

The ability to accurately measure the abundance of nucleic acid molecules is key for a variety of applications, e.g., for the measurement of nucleic acid biomarkers, genomics, non-invasive prenatal testing, nucleic acid-tagged combinatorial chemistry, single cell sequencing and the analysis of proteins using nucleic acid-tagged antibodies. There is a constant need for new methods for measuring the abundance of nucleic acid molecules in a sample.

### SUMMARY

This disclosure provides, among other things, a method of analyzing a sample by hybridizing oligonucleotides in a predefined temporal order. In some embodiments, the method may comprise: sequentially hybridizing multiple sets of barcoded detector oligonucleotides with a sample that comprises a population of target molecules, wherein the detector oligonucleotides are complementary to a sequence in the target molecules and the different sets of oligonucleotides hybridize to the sequence in a predefined temporal order, and wherein the barcodes of the detector oligonucleotides identify the order of hybridization; and quantifying the amount of each barcode sequence in the barcoded detector oligonucleotides that hybridize to the sequence in the population of molecules.

In some embodiments, the method may comprise extending the detector oligonucleotides as they hybridize to the target sequence using the target sequence as a template (e.g., by primer extension or by ligation), thereby increasing the Tₘ of the oligonucleotides and preventing them from disassociating from the target sequence after hybridization. In these embodiments, the method may comprise quantifying the extension products, e.g., by sequencing, qPCR, or by hybridization to an array.

Reagents for performing the method are also provided.

The method finds use in a variety of sample analysis methods. In particular, the method finds use in the analysis of gene expression, molecular screens and for detecting proteins. In these embodiments, the target sequence may be a sequence in a library of guide RNAs, a phage display library, oligonucleotide-tagged combinatorial chemistry library or the like, for example, or a sequence in oligonucleotides that have been cleaved from a binding agent such as an antibody.

### BRIEF DESCRIPTION OF THE FIGURES

The skilled artisan will understand that the drawings, described below, are for illustration purposes only. The drawings are not intended to limit the scope of the present teachings in any way.

FIG. 1 schematically illustrates some principles of the method.

### DEFINITIONS

Before describing exemplary embodiments in greater detail, the following definitions are set forth to illustrate and define the meaning and scope of the terms used in the description.

Numeric ranges are inclusive of the numbers defining the range. Unless otherwise indicated, nucleic acids are written left to right in 5′ to 3′ orientation; and, amino acid sequences are written left to right in amino to carboxy orientation, respectively.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

It must be noted that as used herein and in the appended claims, the singular forms “a”, “an”, and “the” include plural referents unless the context clearly dictates otherwise. For example, the term “a primer” refers to one or more primers, i.e., a single primer and multiple primers. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

The term “nucleotide” is intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups or are functionalized as ethers, amines, or the likes.

The term “oligonucleotide” as used herein denotes a multimer of nucleotides of about 2 to 200 nucleotides, up to 500 nucleotides in length. Oligonucleotides may be synthetic or may be made enzymatically, and, in some embodiments, are 20 to 150 nucleotides in length. Oligonucleotides may contain ribonucleotide monomers (i.e., may be oligoribonucleotides) or deoxyribonucleotide monomers. An oligonucleotide may be 10 to 20, 21 to 30, 31 to 40, 41 to 50, 51 to 60, 61 to 70, 71 to 80, 80 to 100, 100 to 150 or 150 to 200 nucleotides in length, for example. Oligonucleotides may contain nucleotide analogs and modified backbones, for example.

The term “primer” as used herein refers to an oligonucleotide that is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be single-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence or fragment, the oligonucleotide primer typically contains 10-25 or more nucleotides, although it may contain fewer or more nucleotides. The primers herein are selected to be substantially complementary to a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

The term “hybridization” or “hybridizes” refers to a process in which a nucleic acid strand anneals to and forms a stable duplex, either a homoduplex or a heteroduplex, under normal hybridization conditions with a second complementary nucleic acid strand, and does not form a stable duplex with unrelated nucleic acid molecules under the same normal hybridization conditions. The formation of a duplex is accomplished by annealing two complementary nucleic acid strands in a hybridization reaction. The hybridization reaction can be made to be highly specific by adjustment of the hybridization conditions (often referred to as hybridization stringency) under which the hybridization reaction takes place, such that hybridization between two nucleic acid strands will not form a stable duplex, e.g., a duplex that retains a region of double-strandedness under normal stringency conditions, unless the two nucleic acid strands contain a certain number of nucleotides in specific sequences which are substantially or completely complementary. “Normal hybridization or normal stringency conditions” are readily determined for any given hybridization reaction. See, for example, Ausubel et al., Current Protocols in Molecular Biology, John Wiley & Sons, Inc., New York, or Sambrook et al., Molecular Cloning: A Laboratory Manual, Cold Spring Harbor Laboratory Press. As used herein, the term “hybridizing” or “hybridization” refers to any process by which a strand of nucleic acid binds with a complementary strand through base pairing.

A nucleic acid is considered to be “selectively hybridizable” to a reference nucleic acid sequence if the two sequences specifically hybridize to one another under moderate to high stringency hybridization and wash conditions. Moderate and high stringency hybridization conditions are known (see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.). One example of high stringency conditions includes hybridization at about 42 C in 50% formamide, 5×SSC, 5× Denhardt's solution, 0.5% SDS and 100 ug/ml denatured carrier DNA followed by washing two times in 2×SSC and 0.5% SDS at room temperature and two additional times in 0.1×SSC and 0.5% SDS at 42° C.

The term “sequencing”, as used herein, refers to a method by which the identity of at least 10 consecutive nucleotides (e.g., the identity of at least 20, at least 50, at least 100 or at least 200 or more consecutive nucleotides) of a polynucleotide are obtained.

The term “next-generation sequencing” refers to the so-called parallelized sequencing-by-synthesis or sequencing-by-ligation platforms currently employed by, e.g., Illumina, Life Technologies, and Roche etc. Next-generation sequencing methods may also include nanopore sequencing methods or electronic-detection based methods such as, e.g., Ion Torrent technology commercialized by Life Technologies.

The term “duplex,” or “duplexed,” as used herein, describes two complementary polynucleotides that are base-paired, i.e., hybridized together.

The terms “determining,” “measuring,” “evaluating,” “assessing,” “assaying,” and “analyzing” are used interchangeably herein to refer to forms of measurement, and include determining if an element is present or not. These terms include both quantitative and/or qualitative determinations. Assessing may be relative or absolute.

The term “terminal nucleotide”, as used herein, refers to the nucleotide at either the 5′ or the 3′ end of a nucleic acid molecule. The nucleic acid molecule may be in double-stranded form (i.e., duplexed) or in single-stranded form.

The term “ligating”, as used herein, refers to the enzymatically catalyzed joining of the terminal nucleotide at the 5′ end of a first DNA molecule to the terminal nucleotide at the 3′ end of a second DNA molecule.

The terms “plurality”, “multiple” and “population” are used interchangeably to refer to something that contains at least 2 members. In certain cases, a plurality may have at least 10, at least 100, at least 1000, at least 10,000, at least 100,000, at least 10⁶, at least 10⁷, at least 10⁸ or at least 10⁹ or more members. In some cases, a plurality has up to 10, up to 20 or up to 50 members.

An “oligonucleotide binding site” refers to a site to which an oligonucleotide hybridizes in a target polynucleotide or fragment. If an oligonucleotide “provides” a binding site for a primer, then the primer may hybridize to that oligonucleotide or its complement.

The term “separating”, as used herein, refers to physical separation of two elements (e.g., by size or affinity, etc.) as well as degradation of one element, leaving the other intact.

The term “reference chromosomal region,” as used herein refers to a chromosomal region of known nucleotide sequence, e.g., a chromosomal region whose sequence is deposited at NCBI's Genbank database or other databases, for example.

The term “strand” as used herein refers to a nucleic acid made up of nucleotides covalently linked together by covalent bonds, e.g., phosphodiester bonds.

In a cell, DNA usually exists in a double-stranded form, and as such, has two complementary strands of nucleic acid referred to herein as the “top” and “bottom” strands. In certain cases, complementary strands of a chromosomal region may be referred to as “plus” and “minus” strands, the “first” and “second” strands, the “coding” and “noncoding” strands, the “Watson” and “Crick” strands or the “sense” and “antisense” strands. The assignment of a strand as being a top or bottom strand is arbitrary and does not imply any particular orientation, function or structure. The nucleotide sequences of the first strand of several exemplary mammalian chromosomal regions (e.g., BACs, assemblies, chromosomes, etc.) is known, and may be found in NCBI's Genbank database, for example.

The term “extending”, as used herein, refers to the extension of a primer in a template-specific manner by the addition of nucleotides using a polymerase or by the splinted ligation of a first oligonucleotide to a second oligonucleotide, using the template as a splint. If an oligonucleotide that is annealed to a nucleic acid is extended, the nucleic acid acts as a template for an extension reaction, by a polymerase or splinted ligation. In some cases, two oligonucleotides can be ligated together using the template as a splint, and then the 3′ oligonucleotide (now ligated to the 5′ oligonucleotide) is extended.

The term “in series” is intended to refer to steps that are performed one after the other, on the same sample, i.e., not multiple reactions that are performed on multiple aliquots of a sample.

The term “aliquot” is intended to refer to a portion of a composition. An aliquot can be in the range of 0.5 ul to 10 ul, e.g., 1 ul to 5 ul for example, although other volumes can be employed depending on the scale of an experiment.

The term “barcode sequence”, “barcode”, or “molecular barcode” as used herein, refers to a unique sequence of nucleotides that is sufficiently complex to provide information about (e.g., the source of) a sequence that is appended to the barcode. For example, in many embodiments less than 100 sets of detector oligonucleotides may be used in the present method (e.g., up to 10 or up to 20 mixtures) in the method and, as such, the detector oligonucleotides may contain the same number of barcode sequences in order to identify the set in which they belong. Barcode sequences may be error correcting in some embodiments. A barcode may be at least 2 nucleotides in length (e.g., 2-20 nucleotides).

The term “unique molecular identifier” or UMI refers to a sequence that can be used to identify sequence reads that are derived from the same initial molecule. Such a sequence, alone or in combination with other features of a sequence read, can be used to distinguish between the different molecules that input into an amplification reaction, prior to sequencing. The complexity of a population of unique molecule identifier sequences used in any one implementation may vary depending on a variety of parameters, e.g., the number of molecules in an initial sample and/or the amount of the sample that is used in a subsequent step. For example, in certain cases, the unique molecule identifier may be of low complexity (e.g., may be composed of a mixture of 8 to 1024 sequences). In other cases, the unique molecule identifier may be of high complexity (e.g., may be composed of 1025 to 1M or more sequences). For example, a random sequence (or 4-8 nucleotides in length) can be used in some cases. Unique molecule identifiers are described in Casbon et al (Nuc. Acids Res. 2011, 22 e81), among many others.

The term “sample identifier sequence” or “sample index” is a sequence of nucleotides that can be used to identify the source of a target polynucleotide (i.e., the sample from which sample the target polynucleotide is derived). In use, each sample is tagged with a different sample identifier sequence (e.g., one sequence is appended to each sample, where the different samples are appended to different sequences), and the tagged samples are pooled. After the pooled sample is sequenced, the sample identifier sequence can be used to identify the source of the sequences.

The term “hybridizes to” is intended to mean that two sequences have sufficient complementarity to hybridize to form a duplex under the conditions used. In some instances, two sequences that hybridize to one another may have perfect complementarity. In other instances, two sequences that hybridize may have one or more mismatches or other destabilizing features that lower the melting temperature of the duplex. In these embodiments, one of the hybridizing sequences may have a sequence of at least 10, at least 20 or at least 30 contiguous nucleotides that is at least 90% or at least 95% identical to a sequence in the other hybridizing sequence.

The term “variable”, in the context of two or more nucleic acid sequences that are variable, refers to two or more nucleic acids that have different sequences of nucleotides relative to one another. In other words, if the polynucleotides of a population have a variable sequence or a particular sequence “varies”, then the nucleotide sequence of the polynucleotide molecules of the population varies from molecule to molecule. The term “variable” is not to be read to require that every molecule in a population has a different sequence to the other molecules in a population.

The term “target sequence” in the context of a sample that comprises a target sequence, refers to sample that comprises a population of molecules that comprise the target sequence.

The term “concentration” may be relative to something else, absolute or arbitrarily defined (e.g., “10×” or “100×”).

A “set” can be represented by a single member (e.g., a single oligonucleotide) or multiple members, e.g., (up to 10, up to 50 or up to 100 oligonucleotides that hybridize to different target sequences).

The term “sequentially hybridizing” is intended to mean that the oligonucleotides are hybridized to the sample in temporal order, i.e., one after another, without denaturing the sample in between.

The term “predefined temporal order” is intended to mean that the order of hybridization is known beforehand.

The term “extending” is intended include a templated reaction that is catalyzed by a polymerase (i.e., primer extension) or a ligase (splinted ligation).

Other definitions of terms may appear throughout the specification.

### DESCRIPTION OF EXEMPLARY EMBODIMENTS

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

The section headings used herein are for organizational purposes only and are not to be construed as limiting the subject matter described in any way. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, the some exemplary methods and materials are now described.

The citation of any publication is for its disclosure prior to the filing date and should not be construed as an admission that the present claims are not entitled to antedate such publication by virtue of prior invention. Further, the dates of publication provided can be different from the actual publication dates which can need to be independently confirmed.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

All patents and publications, including all sequences disclosed within such patents and publications, referred to herein are expressly incorporated by reference.

### Methods of Sample Analysis

A method of analyzing a sample by hybridizing oligonucleotides in a predefined temporal order is provided. As noted above, in some embodiments, the method may comprise sequentially hybridizing multiple sets of barcoded detector oligonucleotides with a sample in a predefined temporal order. In this method, the barcodes of the detector oligonucleotides identify the order in which the detector oligonucleotides hybridize to the target sequence in the sample. For example, a first set of detector oligonucleotides have a first barcode and are hybridized with the target sequences, then a second set of detector oligonucleotides that have a second barcode are hybridized, then a third set of detector oligonucleotides that have third barcode are hybridized, where the first, second and third barcodes are different and the hybridized detector oligonucleotides are not disassociated from the target sequences in between. In this example, assuming that there is a sufficient number of target molecules in the sample, all molecules of the first set of detector oligonucleotides (which have the first barcode) will hybridize to the target molecules, all molecules of the second set of detector oligonucleotides (which have the second barcode) will hybridize to the available target sequences (i.e., the target sequences that are not already occupied by the first set of detector oligonucleotides), and all molecules of the third set of detector oligonucleotides (which have the third barcode) will hybridize to the available target sequences (i.e., the target sequences that are not already occupied by the first and second sets of detector oligonucleotides). At the end of the reaction, if all three of the barcodes (i.e., the first, second and third barcodes) are represented in the hybridized detector oligonucleotides then there should be more target molecules than the number of detector oligonucleotide molecules in the first and second sets combined. If only two of the barcodes (i.e., the first and second barcodes) are represented in the hybridized detector oligonucleotides then there should be more target molecules than the number of detector oligonucleotide molecules in the first set. If only one of the barcodes (i.e., the first barcode) is represented in the hybridized detector oligonucleotides then there should be the same or less target molecules as the number of detector oligonucleotide molecules in the first set. As would be apparent, the abundance of the population of molecules that comprise the target sequence can be quantified using this method. As such, in some embodiments, the method may comprise sequentially hybridizing multiple sets of barcoded detector oligonucleotides with a sample that comprises a population of target molecules, wherein the detector oligonucleotides are complementary to a sequence in the target molecules and the different sets of oligonucleotides hybridize to the sequence in a predefined temporal order, and wherein the barcodes of the detector oligonucleotides identify the order of hybridization; and (b) quantifying the amount of each barcode sequence in the barcoded detector oligonucleotides that hybridize to the sequence in the population of molecules in the hybridization step.

In some embodiments, the method may comprise extending the detector oligonucleotides as they hybridize to the target sequence using the target sequence as a template, thereby increasing the Tₘ of the oligonucleotides and preventing them from disassociating from the target sequence after they have hybridized to the target sequence. This can be done in a variety of different ways. For example, in some embodiments, the oligonucleotides may be primers that are extended by a polymerase using the target sequence as a template immediately after hybridization of the oligonucleotide to the target sequence. Alternatively, in some embodiments, the oligonucleotides may be primers that ligated to another oligonucleotide using the target sequence as a splint immediately after hybridization of the oligonucleotide to the target sequence. In either of these embodiments, the enzyme used to catalyze the extension of the oligonucleotide (e.g., the polymerase or ligase) may be thermostable. Because the amount of each barcode sequence can be determined by examining the extension products, the method may comprise quantifying the extension products, e.g., by RT-PCR, sequencing or hybridization to an array, etc. In some embodiments, the detector oligonucleotides may have a 5′ tail, thereby allowing the extension products to be amplified and sequenced. For example, if primer extension is used, the extension products may be amplified using a gene-specific primer and a primer that hybridizes to the complement of the tail. If a splinted ligation is used to extend the detector oligonucleotides, then the extension products may be amplified using a primer that hybridizes to a common sequence in the tail in the detector oligonucleotides and a common sequence in the tail in the oligonucleotides to which the detector oligonucleotides are ligated.

The principle described above may be applied to a variety of different reactions. For example, the detector oligonucleotides may hybridize to substantially unique sequences in the sample (i.e., may be “sequence-specific”). In these embodiments, the amount of a target sequence in the sample may be determined because, at some point in time, the detector oligonucleotides will have hybridized to all of the target sequence and extended. In other embodiments, the detector oligonucleotides may have a 3′ end that is oligo-d(T) or a random sequence (e.g., 4-6 nucleotides of N, where N is G, A, T or C). In these embodiments, the detector oligonucleotides should hybridize to the most abundant sequences (since they are most abundant), and the least abundant sequences last, thereby providing a way to quantify the abundance of sequences in the sample. In any embodiment, the target sequence can be DNA or RNA.

In some embodiments, a defined amount (e.g., in the range 100 to 1M or more molecules) of each of the detector oligonucleotides may be hybridized in each hybridization cycle. For example, in some embodiments, the different sets (e.g., the first, second and third sets) of detector oligonucleotides may contain the same amount of detector oligonucleotides. In some embodiments, the different sets (e.g., the first, second and third sets) of detector oligonucleotides may contain different amounts of the detector oligonucleotides. For example, in some embodiments, the first set of detector oligonucleotides may have relatively low amount of detector oligonucleotides (e.g., in the range of 10-500 molecules), the second set of detector oligonucleotides may have a higher amount of detector oligonucleotides (e.g., in the range of 500-10,000 molecules) and the third set of detector oligonucleotides may have the highest amount detector oligonucleotides (e.g., in the range of 10,000-50,000 molecules), and so on. Conversely, in some embodiments, the first set of detector oligonucleotides may have relatively higher amount of detector oligonucleotides (e.g., in the range of 10,000-50,000 molecules), the second set of detector oligonucleotides may have a lower amount of detector oligonucleotides (e.g., in the range of 500-10,000 molecules) and the third set of detector oligonucleotides may have the lowest amount detector oligonucleotides (e.g., in the range of 10-500 molecules), and so on. In these embodiments, the detector oligonucleotides may be hybridized in order of their amount. In some embodiments, the amounts of the detector oligonucleotides in the different sets may be 2-fold to 10-fold different from one another, e.g., may be represented by a series of 2×-10× (e.g., 2×, 5× or 10×) increases in concentration. For example, in some embodiments, the detector oligonucleotides in the first, second, third and fourth oligonucleotide sets may be at a concentration of 1, 10, 100 and 1,000, 1, 2, 4, and 8 or 1, 5, 25 and 125, for example, depending on whether the oligonucleotide sets have a 2-fold, 5-fold or 10-fold difference in concentrations of the detector oligonucleotides.

In some embodiments, the method comprises determining a profile of barcode sequences of the detector oligonucleotides that hybridize to the target sequence in the population of molecules. Illustrated by example, a profile may indicate the number of detector oligonucleotides that hybridize to the target sequence and have the first barcode, the number of detector oligonucleotides that hybridize to the target sequence and have the second barcode and the number of detector oligonucleotides that hybridize to the target sequence and have the third barcode, and so on. The abundance of the population of molecules that comprise the target sequence in the sample can be quantified using the profile of barcode sequences.

As would be apparent from the above, the amount of each barcode sequence in the barcoded detector oligonucleotides that hybridize to the sequence in the population of molecules in the hybridization step may be quantified after the hybridization step has been completed.

The sequential hybridization may be implemented in a variety of different ways. In some embodiments, each set of detector oligonucleotides is a separate mixture. In these embodiments, the method may comprise (a) hybridizing one of the mixtures of detector oligonucleotides to the sample produce a hybridized sample; and, (b) after a period of time (e.g., after a period of time in the range of 10 s-1 hr) and without disassociating the detector oligonucleotides that have already hybridized to the sample, repeating step (a) using a different mixture of detector oligonucleotides, until all of the mixtures of oligonucleotides have been hybridized to the sample. In these embodiments, at least 2, e.g., at least 3, at least 4, at least 5, at least 6, at least 7, at least 8, at least 9, or at least 10 mixtures of oligonucleotides (each having a different barcode) may be hybridized to the sample in order, without disassociating the oligonucleotides that have already hybridized to the target sequence in between each cycle. In other embodiments, the sequential hybridization may be implemented in a single tube reaction, without adding additional reagents during the course of the rejection, i.e., in a “one-pot” reaction. This can be done by progressively lowering the temperature of the hybridization. In this embodiment, the detector oligonucleotides in the first set would have the highest Tₘ, the detector oligonucleotides in the first second would have a Tₘ that is lower than the Tm of the detector oligonucleotides in the first set, and the detector oligonucleotides in the third set would have a Tₘ that is lower than the Tₘ of the detector oligonucleotides in the second set, and so-on. The difference between the Tₘs of the different sets (i.e., the difference between the Tms of the detector oligonucleotides of first and second sets, the detector oligonucleotides of second and third sets, and so on), may be in the range of 3° C. to 10° C. such that the detector oligonucleotides of the different sets can hybridize at different temperatures and the sequential hybridization can be effected by decreasing the temperature of the hybridization. In these embodiments, at least 2, e.g., at least 3, at least 4, at least 5, at least 6, at least 7, at least 8, at least 9, or at least 10 sets of oligonucleotides (each having target complementary sequence having a different Tₘ, and a different barcode) may be hybridized to the sample in order, in a single reaction, by lowering the temperature of the hybridization, without disassociating the oligonucleotides that have already hybridized to the target sequence in between each cycle. In these embodiments, within the oligonucleotides within each set may be Tₘ-matched, where the term “Tₘ-matched” refers to sequences that have melting temperatures that are within a defined range, e.g., less than 2 or 3° C. of a defined temperature. The Tₘs of the detector oligonucleotides in the different sets may be altered by e.g., decreasing the lengths of the hybridizing sequences or including mismatches, changing the backbone (to PNA for example), adding different bases, or other destabilizing features. Across sets, the detector oligonucleotide should share a sequence of at least 10, at least 15, at least 20, at least 25 or at least 30 contiguous nucleotides, such that they bind to the target sequence.

A detector oligonucleotide may be at least 10 nucleotides in length, e.g., at least 15, at least 20 nucleotides, at least 30 nucleotides or at least 40 nucleotides in length, for example. The target-complementary sequence in a detector oligonucleotide may be at least at least 10, at least 15, at least 20, at least 25 or at least 30 nucleotides length. In any embodiment, a detector oligonucleotide a partially double-stranded “toehold” oligonucleotide (see, e.g., Byrom et al, Nucleic Acids Res. 2014 42: e120).

In some embodiments, the method may comprise: (a) obtaining multiple sets of detector oligonucleotides, wherein each set comprises at least a first detector oligonucleotide comprising a first target-complementary sequence and a barcode sequence, wherein the first target-complementary sequence is complementary to a first target sequence and the barcode sequence that varies from set to set; (b) sequentially hybridizing the sets of detector oligonucleotides of (a) with a sample that comprises a population of molecules that comprise the first target sequence, wherein the sets of detector oligonucleotides hybridize to the first target sequence in a predefined temporal order; and (c) quantifying the amount of each barcode sequence in the first detector oligonucleotides that hybridize to the first target sequence in the population of molecules in step (b).

In these embodiments, the multiple sets of detector oligonucleotides may comprise: a first set of detector oligonucleotides comprising a first detector oligonucleotide comprising a first barcode sequence and a sequence that is complementary to the first target sequence; a second set of detector oligonucleotides comprising a first detector oligonucleotide comprising a second barcode sequence and a sequence that is complementary to the first target sequence, and a third set of detector oligonucleotides comprising a first detector oligonucleotide comprising a third barcode sequence a sequence that is complementary to the first target sequence.

In some embodiments, each set of detector oligonucleotides may further comprise a second detector oligonucleotide comprising a second target-complementary sequence and a barcode sequence, wherein the second target-complementary sequence is complementary to a second target sequence and the barcode sequence varies from set to set; wherein the second detector oligonucleotides hybridize to a second target sequence in the sample in a predefined temporal order and wherein step (c) further comprises quantifying the amount of each barcode in the second detector oligonucleotides that hybridize to the second target sequence in the population of molecules in step (b).

In these embodiments, the sets of detector oligonucleotides may comprise: a first set of detector oligonucleotides that comprises (a) a first detector oligonucleotide comprising a barcode sequence and a sequence that is complementary to the first target sequence and (b) a second detector oligonucleotide comprising a barcode sequence and a sequence that is complementary to a second target sequence; a second set of detector oligonucleotides that comprises (a) a first detector oligonucleotide comprising a barcode sequence and a sequence that is complementary to the first target sequence and (b) a second detector oligonucleotide comprising a barcode sequence and a sequence that is complementary to the second target sequence, and a third set of detector oligonucleotides that comprises (a) a first detector oligonucleotide comprising a barcode sequence and a sequence that is complementary to the first target sequence and (b) a second detector oligonucleotide comprising a barcode sequence and a sequence that is complementary to the third target sequence; wherein the barcode sequences in the first, second and third sets are different.

In some embodiments, the sequence that is complementary to the first target sequence is the same in all of the first detector oligonucleotides. In other embodiments, the sequences that are complementary to the first target sequence in the first detector oligonucleotides hybridize the first target sequence with different Tₘs.

### Reagents

Also provided herein is a reagent system. In some embodiments, the system may comprise multiple sets (e.g., at least 2, at least 3, at least 4, at least 5, up to 10 or 20 or more sets) of detector oligonucleotides, wherein each set comprises at least a first detector oligonucleotide comprising a barcode sequence and a first target-complementary sequence, wherein: the first target-complementary sequence is complementary to a first target sequence; the barcode sequence that varies from set to set; and the first detector oligonucleotides all hybridize to the same target sequence but at different Tₘs. In many embodiments, the sets of detector oligonucleotides are mixed together, i.e., in the form of an aqueous or dried mixture comprising the sets of detector oligonucleotides.

In some embodiments, the reagent system may comprise a first set of detector oligonucleotides comprising a first detector oligonucleotide comprising a first barcode sequence and a sequence that is complementary to the first target sequence; a second set of detector oligonucleotides comprising a first detector oligonucleotide comprising a second barcode sequence and a sequence that is complementary to the first target sequence, and a third set of detector oligonucleotides comprising a first detector oligonucleotide comprising a third barcode sequence and a sequence that is complementary to the first target sequence; wherein the first detector oligonucleotides in the first, second and third sets hybridize to the same target sequence but at different Tₘs.

In some embodiments, each set of detector oligonucleotides may further comprise a second detector oligonucleotide comprising: (i) a sequence that is complementary to second target sequence; and (ii) a barcode sequence that varies from set to set; wherein all of second detector oligonucleotides hybridize to the second target sequence but at different Tₘs. In these embodiments, the detector oligonucleotides may be Tₘ matched within each set. In these embodiments, the reagent system may comprise a first set of detector oligonucleotides that comprises (a) a first detector oligonucleotide comprising a barcode sequence and a sequence that is complementary to the first target sequence and (b) a second detector oligonucleotide comprising a barcode sequence and a sequence that is complementary to a second target sequence; a second set of detector oligonucleotides that comprises (a) a first detector oligonucleotide comprising a barcode sequence and a sequence that is complementary to the first target sequence and (b) a second detector oligonucleotide comprising a barcode sequence and a sequence that is complementary to the second target sequence, and a third set of detector oligonucleotides that comprises (a) a first detector oligonucleotide comprising a barcode sequence and a sequence that is complementary to the first target sequence and (b) a second detector oligonucleotide comprising a barcode sequence and a sequence that is complementary to the second target sequence, wherein the barcode sequences in the first, second and third sets are different, the sequences that are complementary to the first and second target sequences are Tm-matched within the sets, the Tₘs at which the sequences that are complementary to the first and second target sequences are Tm-matched within the sets, but different between the sets.

Further detail of the detector oligonucleotides may be found in the methods section above. As explained in greater detail above, the oligonucleotide sets may be in separate containers (e.g., as separate mixtures, where each container contains an oligonucleotide set), or all mixed together in the same container. In embodiments in which mixtures of the different oligonucleotide sets are in separate containers, the detector oligonucleotides in the different containers can have the same sequence with the exception of the barcode. In embodiments in which the different oligonucleotide sets are mixed together in the same container, the sequences that hybridize to the target sequence in the different oligonucleotide sets may be different in order to allow the different sets of oligonucleotides to hybridize to the target in order by, for example, lowering the temperature of the hybridization. As such, in some embodiments, the target-complementary sequences in the detector oligonucleotides in some sets may be longer or shorter than others, for example.

The lengths of the any required sequences may vary independently. In some embodiments, the target sequences (or complements thereof) may be at least 8 nucleotides in length, e.g., 10-20 nucleotides in length, whereas barcode n can be as short as a single base and as long as needed. In typical embodiments, the barcode sequences are 2-10 nucleotides in length.

In some embodiments, the detector oligonucleotide(s) may incorporate one or more primer binding sites, such that certain products can be amplified after a reaction has occurred.

In some embodiments, the target sequences to which the detector oligonucleotides hybridize may be a biological sequence (e.g., RNA, cDNA or genomic DNA, etc.) from any species such as a microorganism, a plant or an animal, such as a mammal. In other embodiments, the target sequence may be non-biological. In these embodiments, the detector oligonucleotide may hybridize to a synthetic oligonucleotide, for example,

The detector oligonucleotide may comprise a UMI (a unique molecule identifier) that, in addition to the barcode, can be used to quantify target molecules. In general, the detector oligonucleotide(s) (and their barcodes) are designed to minimize cross-hybridization with each other.

In some embodiments, the detector oligonucleotide sets may be multiplexed so that the abundance of multiple target sequences (e.g., at least 2, at least 5, at least 10, at least 50, up to 100, 500 or 1,000 or more) can be detected. In these embodiments, the first set of detector oligonucleotides may contain multiple detector oligonucleotides, where each detector oligonucleotide in the set hybridizes to a different target sequence, the second set of detector oligonucleotides may contain a corresponding number of detector oligonucleotides, where the detector oligonucleotides in the second set hybridize to the same target sequence as the first set, and the third set of detector oligonucleotides may contain a corresponding number of detector oligonucleotides, where the detector oligonucleotides in the third set hybridize to the same target sequence as the first and second sets, etc.

In many embodiments, there are less than 100 oligonucleotide sets (e.g., up to 10 or up to 20 oligonucleotide sets) in a system. The oligonucleotide sets may be aqueous or dried, for example. The various sets may be in separate containers or mixed together. The concentration of each detector oligonucleotide in an oligonucleotide set may be in the range of 0.001 to 10 pM, e.g., 10 fM to 5 pM, although concentrations outside of this range can be used in many applications.

In any embodiment, at least the detector oligonucleotide in each set may be a partially double-stranded toehold oligonucleotide (see, e.g., Byrom et al, Nucleic Acids Res. 2014 42:e120).

### Utility

The present method may be used in a wide variety of applications including, but not limited to the analysis of nucleic acid biomarkers, genomics, non-invasive prenatal testing, nucleic acid-tagged combinatorial chemistry, single cell sequencing and the analysis of proteins using nucleic acid-tagged antibodies. In one example, the method can be used to analyze RNA abundance. In these embodiments, the detector oligonucleotides hybridize to RNAs. Any biological sample could be analyzed using the method described above, including DNA/RNA from prokaryotes and eukaryotes, including yeast, plants and animals, such as fish, birds, reptiles, amphibians and mammals. In certain embodiments, the DNA or RNA may be from mammalian cells, i.e., cells from mice, rabbits, primates, or humans, or cultured derivatives thereof. In other embodiments, the method can be used to the abundance of synthetic oligonucleotides. In one example, the method may comprise binding antibodies that are linked to synthetic oligonucleotides to a sample (e.g., serum proteins or a tissue section), washing the sample to remove unbound antibodies, releasing the oligonucleotides, and then quantifying the released oligonucleotides using the method. As such, the present method may be used to quantify the abundance of proteins (if the target sequences are initially conjugated to antibodies that bind to those proteins).

### Kits

Also provided by this disclosure are kits that contain the reagents, as described above. In addition to the above-mentioned components, the subject kit may further include instructions for using the components of the kit to practice the present method.

### EXAMPLES

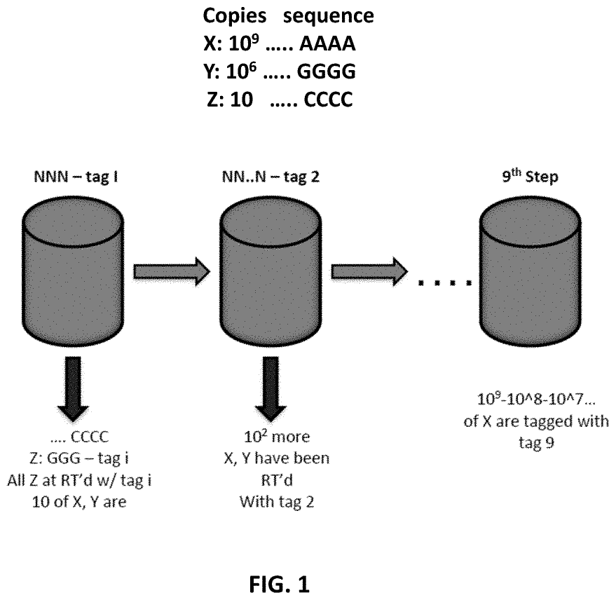

In the illustrative example shown in FIG. 1, X, Y, Z are RNA in a pool P. Consider priming with RT using a sequential pool of tags. All have sequences that are specific for a target at the 3′ end; they differ in a 5′ tag that will encode information: tag 1, tag 2, . . . . Consider sequential RT of the pool with limiting amounts of each pool of tag: at each step, 10 of NNN-tag i are added. The process involves adding a sequence of tagged primers with the abundance of the i^{th} tag at level a: Suppose a₁ . . . aₙ=1/n and aₙ₊₁, . . . aₘ=1/n. If n=10, this corresponds to an expected primary in populations S₁, by only a₁ . . . a₁₀ and of S₂ by a₁₁, . . . a₃₀. Consider simple random sampling (SRS) of the primed pool. Out of every 2 molecules sampled from S₂ will provide information that this molecule has abundance 2× compared to one in S₁. At a sampling depth of I, we therefore compress the variance of estimates of the abundance in each molecule in population I and 2. The method also allows specific retrieval and modulating sample of every strata of tag. DNA can be tagged using random or sequence-specific sets of primers, or sequential splinted ligation: essentially any sequence or semi-sequence specific tagging approach. Consider sampling when a molecule has 1 or 2 counts. If a molecule is from S₁, its tag will always be “1. If from S₂, it has a 0.5 chance of having tag I or tag 2. Thus, if K (identical) counts are observed from a simple sample, there is no way to distinguish whether the molecule is from S₁ or S₂. However, if we use this method, if one has K counts from S₂, the probability that we misidentify it as being from S₁ is (½)^{k}. This shows that the variance of sampling after completion of the method is reduced compared to simple sampling and generalizes to general {aᵢ} designs.

What is claimed is:

1. A method of analyzing a sample by hybridizing oligonucleotides in a predefined temporal order, comprising:
(a) sequentially hybridizing multiple sets of barcoded detector oligonucleotides with a sample that comprises a population of target molecules, wherein the detector oligonucleotides are complementary to a sequence in the target molecules and the different sets of oligonucleotides hybridize to the sequence in a predefined temporal order, and wherein the barcodes of the detector oligonucleotides identify the order of hybridization; and
(b) quantifying the amount of each barcode sequence in the barcoded detector oligonucleotides that hybridize to the sequence in the population of molecules in step (a).

2. The method of claim 1, comprising extending the detector oligonucleotides as they hybridize to the target sequence using the target sequence as a template, thereby increasing the Tₘ of the oligonucleotides and preventing them from disassociating from the target sequence after hybridization.

3. The method of claim 2, wherein the extending is done by primer extension using a thermostable polymerase using the target sequence as a template.

4. The method of claim 2, wherein the extending is done by ligating a second oligonucleotide to the detector oligonucleotides using the target sequence as a ligation splint.

5. The method of claim 2, wherein step (c) comprises quantifying the extension products.

6. The method of claim 1, wherein the quantifying of step (c) is done by sequencing, qPCR, or by hybridization to an array.

7. The method of claim 1, wherein the method comprises determining a profile of barcode sequences of the detector oligonucleotides that hybridize to the target sequence in the population of molecules in step (b).

8. The method of claim 7, wherein the method comprises quantifying the abundance of the population of molecules that comprise the target sequence in the sample based on the profile of barcode sequences.

9. The method of claim 1, wherein each set of detector oligonucleotide is a separate mixture, and the method comprises:
(a) hybridizing a mixture of detector oligonucleotides to the sample produce a hybridized sample; and,
(b) after a period of time, repeating step (a) using a different mixture of detector oligonucleotides, until all of the mixtures of oligonucleotides have been hybridized to the sample.

10. The method of claim 1, wherein the detector oligonucleotides in the different sets hybridize to the same target sequence, but with different Tₘs.

11. The method of claim 10, wherein:
all of the sets of detector oligonucleotide are mixed together with the sample in a reaction mix, and
the sequences that are complementary to the target sequence in the competitor oligonucleotides in the different sets hybridize to the target sequence in order of their Tₘs.

12. The method of claim 11, wherein the sequential hybridization of the sets of detector oligonucleotides is done by lowering the temperature of the hybridization.

13. The method of claim 1, wherein the detector oligonucleotides in the different sets of detector oligonucleotides are hybridized to the sample at the same concentration.

14. The method of claim 1, wherein the detector oligonucleotides in the different sets of detector oligonucleotides are at different concentrations.

15. The method of claim 14, wherein the detector oligonucleotides are hybridized in order or their concentration.

16. The method of claim 1, comprising:
(a) obtaining multiple sets of detector oligonucleotides, wherein each set comprises at least a first detector oligonucleotide comprising a first target-complementary sequence and a barcode sequence, wherein the first target-complementary sequence is complementary to a first target sequence and the barcode sequence that varies from set to set;
(b) sequentially hybridizing the sets of detector oligonucleotides of (a) with a sample that comprises a population of molecules that comprise the first target sequence, wherein the sets of detector oligonucleotides hybridize to the first target sequence in a predefined temporal order; and
(c) quantifying the amount of each barcode sequence in the first detector oligonucleotides that hybridize to the first target sequence in the population of molecules in step (b).

17. The method of claim 1, wherein the plurality of sets of detector oligonucleotides comprises:
a first set of detector oligonucleotides comprising a first detector oligonucleotide comprising a first barcode sequence and a sequence that is complementary to the first target sequence;
a second set of detector oligonucleotides comprising a first detector oligonucleotide comprising a second barcode sequence and a sequence that is complementary to the first target sequence, and
a third set of detector oligonucleotides comprising a first detector oligonucleotide comprising a third barcode sequence and a sequence that is complementary to the first target sequence.

18. The method of claim 17, wherein each set of detector oligonucleotides further comprises a second detector oligonucleotide comprising a second target-complementary sequence and a barcode sequence, wherein the second target-complementary sequence is complementary to a second target sequence and the barcode sequence varies from set to set;
wherein the second detector oligonucleotides hybridize to a second target sequence in the sample in a predefined temporal order and
wherein step (c) further comprises quantifying the amount of each barcode in the second detector oligonucleotides that hybridize to the second target sequence in the population of molecules in step (b).

19. The method of claim 17, wherein the plurality of sets of detector oligonucleotides comprises:
a first set of detector oligonucleotides that comprises (a) a first detector oligonucleotide comprising a barcode sequence and a sequence that is complementary to the first target sequence and (b) a second detector oligonucleotide comprising a barcode sequence and a sequence that is complementary to a second target sequence;
a second set of detector oligonucleotides that comprises (a) a first detector oligonucleotide comprising a barcode sequence and a sequence that is complementary to the first target sequence and (b) a second detector oligonucleotide comprising a barcode sequence and a sequence that is complementary to the second target sequence, and
a third set of detector oligonucleotides that comprises (a) a first detector oligonucleotide comprising a barcode sequence and a sequence that is complementary to the first target sequence and (b) a second detector oligonucleotide comprising a barcode sequence and a sequence that is complementary to the third target sequence;
wherein the barcode sequences in the first, second and third sets are different.

20. The method of claim 16, wherein the sequence that is complementary to the first target sequence of (a)(i) is the same in all of the first detector oligonucleotides.
